(19) United States
(12) Patent Application Publication
(10) Pub. No.: US 2004/0181433 A1
(43) Pub. Date: Sep. 16, 2004

(54) Patient compliance and follow-up techniques

(76) Inventor: David J. Blair, Fishers, IN (US)

Correspondence Address: Woodard, Emhardt, Moriarty, McNett & Henry LLP, Bank One Center/Tower, Suite 3700, 111 Monument Circle, Indianapolis, IN, 46205-5137, US

(21) Appl. No.: 10/798,048

(22) Filed: Mar. 11, 2004

Related U.S. Application Data
(60) Provisional application No. 60/453,657, filed on Mar. 11, 2003.

(51) Int. Cl. G06F 17/60; A61B 5/00

(52) U.S. Cl. 705/2

(57) ABSTRACT

One embodiment of the present invention includes a monitoring service that contacts patients who have recently visited a health care provider. This service gathers post-visit information from the patient to improve patient compliance with the health care provider's instructions, provide better patient outcomes, increase patient satisfaction with their health care, and lower health care costs. In one form, the service utilizes pharmacists to contact patients as part of the monitoring service. Alternatively or additionally, data relating to the monitoring service is selectively collected and disseminated through a computer network.

### CROSS-REFERENCE TO RELATED APPLICATIONS

The present application claims the benefit of U.S. Provisional Patent Application No. 60/453,657 filed 11 Mar. 2003, which is hereby incorporated by reference in its entirety.

### BACKGROUND

The present invention relates generally to a system and method for evaluating patient compliance with medical advice and a system for follow-up on a patient's condition. While the system and method has various widespread applications, it is particularly suited for evaluating compliance with medications prescribed or authorized by a health care service provider.

In many instances, the quality of health care afforded a given patient could be improved by better monitoring of the patient's understanding of the treatment regimen, the patient's compliance with medications, and the efficacy of the treatment regimen. Unfortunately, patient monitoring is often the weakest link in the patient-management chain. Busy physicians and their staff may not have time to follow-up with every patient for whom a treatment regimen has been prescribed. Thus, the burden of reporting adverse reactions, ineffective treatment, or confusion over instructions falls upon the patient. Frequently, patients do not report these experiences to their physicians, and simply stop following the prescribed treatment regimen.

Thus, there remains a need for further advancement in this area. The present invention satisfies this a need.

### SUMMARY

One embodiment of the present invention includes a unique technique to monitor patients. Other embodiments of the present invention include unique methods, systems, devices, and apparatus to monitor compliance and/or follow-up with patients after visiting a health care provider.

A further embodiment of the present invention includes contacting a patient after a visit to a health care provider to obtain post-visit information and entering this information in a patient information database through a computer network. At least a portion of the post-visit information is provided to the health care provider from the database after entry.

In another embodiment, a server for storing a patient information database is provided. One or more patient contacts are assigned to a health care monitor through a computer network coupled to the server. The server is programmed to provide these assignments. A patient response is obtained by the health care monitor, is received through the computer network, and is stored in the patient information database. Data about the response is sent to a health care provider from this database.

Yet a further embodiment of the present invention includes providing a server to store a patient information database and assign a patient contact to a health care monitor. The assignment is sent to the health care monitor through a computer network coupled to the server. Patient data is received through the computer network from the health care monitor in response to the patient contact by the health care monitor. The patient data is stored in the patient information database and is thereafter sent to a health care provider for the patient.

In another embodiment, a computer-accessible device carries programming instructions executable by a computer to perform the method of: generating an assignment of a patient contact for a health care monitor from a patient information database maintained by a server, sending the assignment to the health care monitor through a computer network coupled to the server, receiving patient data through the computer network from the health care monitor, storing the patient data received from the health care monitor in the patient information database, and sending the patient data to a health care provider.

Still a further embodiment of the present invention includes a system having a server operable to maintain a patient information database, an administrative browser site, one or more practitioner browser sites, one or more health care monitor browser sites, and a computer network coupling these together. The server is operable to assign one or more patient contacts to a health care monitor through the computer network, receive a patient response obtained by the health care monitor through the computer network, store the patient response in the patient information database, and send data about the patient response to a practitioner.

For another embodiment, the health care monitor of the previously described embodiments is a pharmacist and/or the patient is contacted by the health care monitor by telephone.

One object of the present invention is to provide a unique technique for patient monitoring.

Another object of the present invention is to provide a unique method, system, device, or apparatus for monitoring patients after visiting a health care provider.

Further objects, embodiments, forms, features, aspects, advantages, and benefits of the present invention shall become apparent from the detailed description and drawing contained herein.

### BRIEF DESCRIPTION OF VIEWS OF THE DRAWING

FIG. 1 is a diagrammatic view of a system of one embodiment of the present invention.

FIG. 2 is a view of a flow diagram of one embodiment of a process that can be performed with the system of FIG. 1.

### DETAILED DESCRIPTION

While the present invention may be embodied in many different forms, for the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

Among the embodiments of the present invention is a monitoring service that contacts patients who have recently visited their doctor. This service gathers post-visit information from the patient to improve patient compliance with doctor's instructions, provide better patient outcomes, increase patient satisfaction with their health care, and lower health care cost. In one form, the service employs pharmacists that contact patients to follow-up on medical treatment. A centralized database is provided to collect medical information about the patient that can be updated as follow-up contacts are made. Physicians and/or other health care workers can access the patient information remotely via a communication network, such as the internet, subject to appropriate security and privacy measures. The service can be sponsored by a specific party, such as a drug manufacturer, insurance company, or other organization interested in a given group of patients.

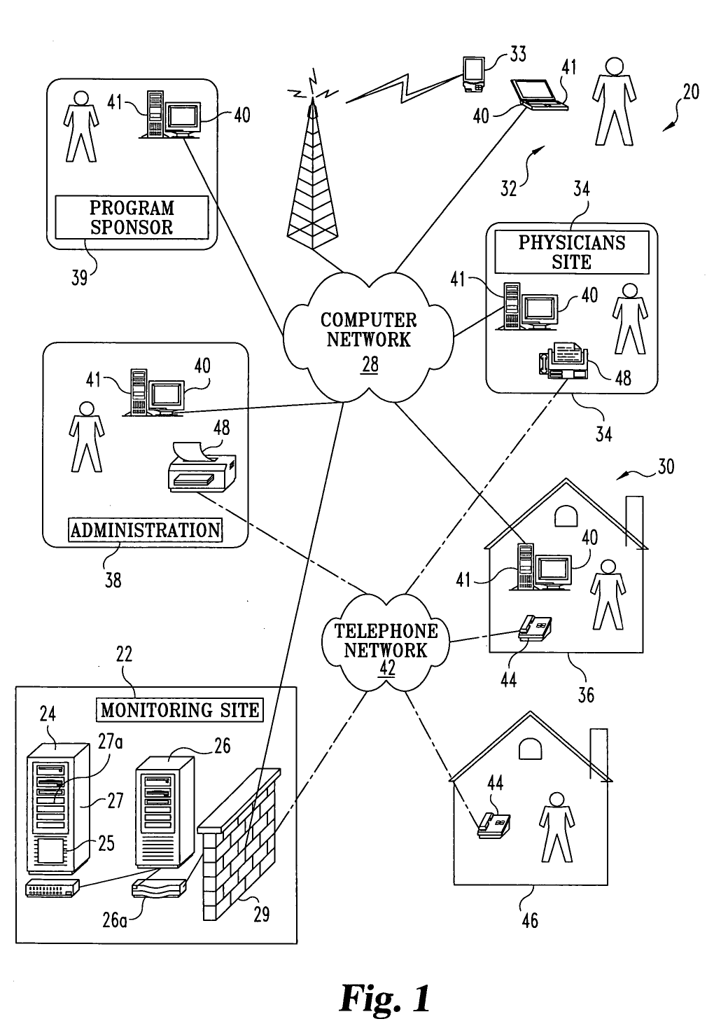

FIG. 1 illustrates system 20 for gathering and maintaining information. System 20 includes a monitoring server site 22 including website server 24 and net server 26, which are operatively coupled together. Website server 24 contains database 25 in which pertinent patient information is stored. Net server 26 is further coupled to computer network 28 through firewall 29. Firewall 29 is coupled to net server 26 via a router to provide network security. As illustrated, servers 24 and 26 are provided as separate computer units interconnected by hub 26a.

Website server 24 is provided with computer 27. Computer 27 includes logic to perform operations associated with various embodiments of the present invention to be more fully described hereinafter. This logic can be in the form of programming instructions, firmware, dedicated hardware, one or more signals carried on a transmission medium, such as computer network 28, and/or different forms as would occur to those skilled in the art. In one embodiment, computer 27 is in the form of a standard type, being at least partially programmable and arranged to execute stored software instructions with one or more semiconductor integrated circuits.

Computer 27 for server 24 includes removable memory device 27a which can be in the form of a tape, cartridge, or disc comprising a non-volatile type of memory. In one form, removable memory device 27a is a floppy or optical disc that can be selectively installed and removed from a corresponding disc drive included in computer 27. In other embodiments, removable memory device 27a may not be present. As an alternative or addition to removable memory device 27a, computer 27 can include one or more other memories as would occur to those skilled in the art, such as solid-state electronic memory, magnetic memory, and/or optical memory. This memory can be volatile, non-volatile, or a combination of both. Indeed, in one form, database 25 resides at least partially on a permanently installed form of memory associated with computer 27, such as one or more electromagnetic hard discs and/or other appropriate mass memory device types. Net server 26 can likewise be provided with a computing device configured like computer 27. Alternatively, servers 24 and 26 can be provided by a common computer unit or with more than two computer units locally or remotely located relative to one another.

Computer network 28 operatively couples a number of remote sites 30 to monitoring server site 22. Representative remote sites 30 include wireless mobile browser site 32 in the form of a Personal Digital Assistant (PDA) 33, physician office site 34, patient health care monitor site 36, and monitoring service administration site 38. A program sponsor site 39 is also illustrated in FIG. 1. Program sponsor site 39 could be a drug company, an insurance company, health care provider network, or other organization. Monitoring server site 22 and/or administration site 38 can control access to database 25 by the other sites 30.

Each site 32, 34, 36, 38, and 39 includes a web browser 40 executed by a computer workstation 41 coupled to computer network 28. Generally, workstations 41 are configured to operate as clients of website server 24, and optionally may be utilized in other computer operations. Workstation 41 can include one or more software programmable processors, one or more input devices, and/or one or more output devices. Input devices can include a conventional mouse and keyboard; and alternatively or additionally may include a track ball, light pen, voice recognition subsystem, or such different input devices as would occur to those skilled in the art. Output devices can include a conventional graphic display device and/or printer, and additionally may include an aural output system, or such different output devices as would occur to those skilled in the art. Computer network 28 includes the internet, and can further include one or more Local Area Networks (LANs) and/or other Wide Area Networks (WANs) besides the internet. In other embodiments, computer network 28 may be of a dedicated type without the internet, may be of another network configuration with or without wireless and/or hardwired communication links, a combination of these, or of a different data communication arrangement as would occur to one skilled in the art.

System 20 also includes a telephone communication network 42 that interconnects telephone communication device 44 at a representative patient site 46 with a telephone communication device 44 at patient health care monitor site 36. Network 42 also couples telefacsimile (fax) devices 48 at sites 34 and 38 together. It should be understood that typically there could be several mobile browser sites 32, physician office sites 34, patient health care monitor sites 36, program sponsor sites 39, and patient sites 42 remotely located relative to one another. Likewise, more than one workstation 41, telephone communication device 44, and/or telefacsimile device 48 could be included at any of sites 30. Additionally or alternatively, there may be more than one monitoring server site 22 and/or administration site 38; however, typically there will only be one of each. In one form, monitoring server site 22 and administration site 38 are combined at a single location, with a LAN coupling computer workstation 41 of administrative site 38 to server 24 via the illustrated hub 26a behind firewall 29. Additionally, one or more remote browser sites 40 could be utilized for administration that interconnect to website server 24 through firewall 29 as illustrated for site 38. In still other embodiments, program sponsor site(s) 39 may be absent, with sponsor communication, if any, being received through administration site 38. Computer network 28 and telephone communication network 42 can be separate from one another or at least partially combined in various embodiments. In one such combination, the Public Switch Telephone Network (PSTN) is at least partially utilized in both networks 28 and 42, and/or a common communication network is used for both networks 28 and 42.

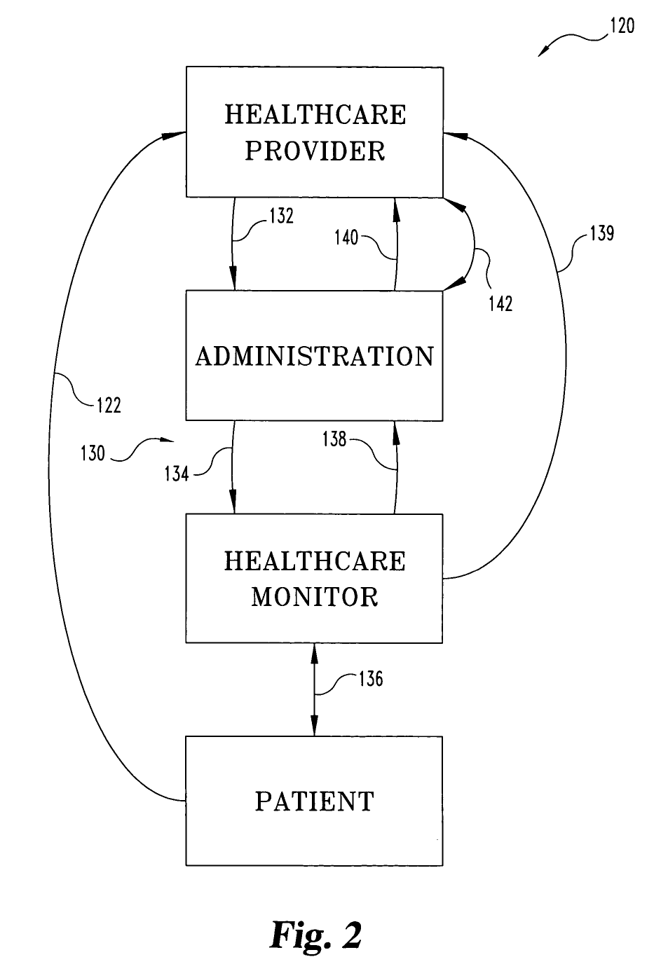

Referring additionally to FIG. 2, a flow diagram for patient monitoring process 120 is illustrated. Process 120 is performed with system 20; where appropriate stages, tests, and/or operations are executed according to the logic associated with computer 27. In FIG. 2, various arrows labeled by reference numerals represent different operations of process 120. In operation 122 of process 120, a patient visits a physician at site 34 for treatment. Alternatively, the visit could be to a hospital or clinic, and/or to see a different type of health care provider such as, a dentist, a practical nurse, or other kind as would occur to one skilled in the art. The health care provider makes a diagnosis and issues a medical treatment regimen to the visiting patient. In the case of a typical visit to a medical doctor, this treatment often includes issuance of a prescription for a medication. If the health care provider determines that a follow-up call to the patient is warranted, then the monitoring service 130 represented by operations 132-142 is activated. Monitoring service 130 is typically executed under an identified program that has a particular sponsor. More than one sponsored program may utilize process 120 with system 20, and a given sponsor may be associated with more than one program. If there is no request to utilize monitoring service 30, process 120 halts with respect to the given patient visit.

In operation 132 of process 120, the health care provider requests a follow-up call be made to the patient. This request can be provided by filling out a patient follow-up form, which contains patient information, along with the diagnosis, drugs prescribed, and other notes. In one embodiment, this form is transmitted from physician office site 34 to administration site 38 by fax via telephone communication network 42. Alternatively or additionally, this information and request could be sent to site 38 via computer network 28, with appropriate security measures being taken. A system administrator enters the patient information from the follow-up request into database 25 with web browser 40 at administration site 38 via computer network 28. If diagnosis and drug codes are used, the administrator converts the diagnosis and drug(s) into appropriate codes and enters these codes into system. If this is the first time the patient has been monitored, a new patient record is created in database 25 by the administrator from site 38. The follow-up call is then assigned to a health care monitor in accordance with logic of web server 24 and/or manually by an administrator at site 38. For the situation where the medical treatment includes the prescription of one or more drugs, a pharmacist is typically assigned the follow-up call.

In operation 134, the assigned health care monitor views the phone call list with web browser 40 at site 36 via computer network 28. For each assigned call, the health care monitor reviews the patient information, patient history, information about the patient's visit, and any questions that should be asked of the patient. Such questions and/or other information may vary with the particular program under which monitoring service 130 is being performed.

In operation 136, the health care monitor at site 36 calls the patient at patient site 46 via telephone communication network 42 and telephone communication device 44. The health care monitor asks the assigned questions, answers questions asked by the patient, and provides counseling. The health care monitor can take other actions noted by the health care provider if the patient's condition has not improved. The patient answers the questions, asks questions, raises issues, and receives counseling and instructions from the pharmacist. In one embodiment there is a maximum number of attempts made to call the patient. If the patient cannot be reached after this maximum number of attempts are made, the contact is considered to be complete. Otherwise, the assigned health care monitor keeps trying to call the patient until the contact is made, or the patient contact assignment is removed.

In operation 138, the health care monitor records the patient outcome in database 25 via computer network 28, including the patient's responses to any questions. The health care monitor also records notes about any counseling provided to the patient, actions taken, and additional follow-up action recommended or needed. In the case of urgent situations, the health care monitor can contact the health care provider directly to initiate action believed to be immediately needed. This optional operation is represented by the process arrow labeled with reference numeral 139.

In operation 140, the patient outcome results are faxed to the corresponding health care provider site 34. The fax can be generated and sent automatically in accordance with programming of server 24 and/or 26 from site 22, or sent from administration site 38. The health care provider reviews the patient outcome and takes action when required. Alternatively or additionally, the health care provider can access his or her patient records in database 25 via computer network 28 to view the outcome and history. This type of operation is represented by the process arrow labeled with reference numeral 142. As an alternative or addition to fax transmission, computer data transmission via computer network 28 and/or voice transmission via telephone communication network 42 can be utilized in other embodiments. In one alternative embodiment, information concerning a patient is entered into database 25 from heath care provider site 34 via computer network 28 without passing through administration site 38. Process 120 continues for each patient until monitoring service 130 is terminated.

Process 120 operates through a number of different groups of individuals that can access at least a portion of database 25: (a) administrators of monitoring service administration site 38, (b) health care monitors corresponding to one or more sites 36, (c) health care providers corresponding to one or more sites 32 or 34 and/or their practices, and (d) one or more program sponsor sites 39. Permissions to restrict or limit access to database 25 can be included in the programming of web server 24 and/or net server 26.

Administrators can be responsible for maintaining master data, creating tasks, and assigning tasks under process 120. Administrators can also define the valid values for certain codes entered in database 25, such as the level of security a given individual/group has with regard to database access. Administrative maintenance of data can include: (a) adding new users, maintaining user information, and/or changing a user's password (User Maintenance); (b) assigning a security role to a new user (Security by User Maintenance); (c) adding a new health care provider practice or updating practice information (Practice Maintenance); (d) adding a new practice location or updating existing location information (Practice Location Maintenance); (e) adding a new practitioner (health care provider) or maintaining existing practitioner information (Practitioner Maintenance); (f) associating a practitioner with a practice (Practice/Practitioner Maintenance); (g) adding a new program sponsor or updating program sponsor information (Sponsor Maintenance); (h) adding a new program, or updating program information (Program Maintenance); (i) adding a patient question to a program, or maintaining existing questions (Program Question Maintenance); (j) adding a new health care monitor or updating monitor information (Monitor Maintenance); and/or (k) associating a health care monitor with a program (Monitor/Program Maintenance). Ongoing activities that an administrator might perform as required in the execution of process 120 include: (a) signing-in (Sign-in screen), (b) accepting terms and conditions of system usage (Legal Notice screen); (c) adding a new patient and maintaining patient information (Patient Maintenance); (d) assigning a patient to a program and maintaining “consent to release” information (Patient/Program Maintenance); (e) adding a new intervention (following a patient's visit to a practitioner), which might be triggered by the receipt of a follow-up request sent from a practice (Intervention Maintenance); (f) reviewing and editing contact outcome (Contact Outcome Maintenance); (g) reviewing call history (Call History Inquiry, Call Outcome Inquiry); (h) maintaining health care monitor schedules (Monitor Schedule Maintenance); (i) assigning contacts to health care monitors (Assign Contact Process); (j) unassigning contacts from health care monitors (Unassign Contact Process); (k) printing a billing report for a program sponsor (Program Billing Report Criteria screen); and/or (l) printing a pharmacist payment report—shows who to pay and how much (Monitor Payment Report Criteria screen).

Under process 120, health care monitors can access selected patient information via computer network 28, review a list of assigned calls, and enter the results of those calls. Typically, a health care monitor is a pharmacist working from a remote site, such as their office or home. Activities that a health care monitor might perform under process 120 include: (a) signing-in (Sign-in screen); (b) accepting terms and conditions of system usage (Legal Notice screen); (c) viewing a list of assigned calls (Home Page); (d) reviewing a list of prior calls made to a patient (Patient Call History List); (e) reviewing the results of a prior call made to a patient (Call Outcome screen); and/or (f) entering the results of a call to a patient (Contact Outcome screen).

Health care providers (practitioners) participating in process 120 can also access the patient information via computer network 28, and review patient outcome history. Typically, the health care provider is a doctor or doctor's assistant working from a remote location, such as their home or office. Activities that a health care provider might perform under process 120 include: (a) signing-in (Sign-in screen); (b) accepting terms and conditions of system usage (Legal Notice screen); (c) choosing a practice (Practice List screen); (d) selecting an inquiry (Home Page); (e) locating a patient in the system (Patient Inquiry); (f) viewing a list of calls made to a patient (Outcome List); and/or (g) viewing an outcome (the results of a call to a patient) (Outcome Results screen).

When utilized, program sponsors can also access selected patient information via computer network 28 to review patient outcome history. In one embodiment, patient information made available to a program sponsor is “sanitized” to remove patient identifiers, such as the patient's name, phone number, etc. A system-assigned identifier can be used to track the same anonymous patient for such embodiments. Activities a program sponsor might perform under process 120 include: (a) signing-in (Sign-in screen); (b) accepting terms and conditions of system usage (Legal Notice screen); (c) choosing a program (Program List screen); (d) selecting an inquiry (Home Page); and/or (e) selecting a report (Home Page). It should be understood that the particular tasks and parties can vary from one embodiment to the next as would occur to those skilled in the art.

In one embodiment, process 120 is implemented using Microsoft Internet Explorer 5.0 compatible browsers. The corresponding website server 24 includes: Microsoft Windows 2000 Advanced Server; Internet Information Service (US) 5.0 for site security, directories, and other configurations; SQL Server 2000 to house and manage database 25; Simple Mail Transfer Protocol (SMTP) services with Collaborative Data Objects (CDO); and Microsoft COM+ Application Server to manage web site Dynamic Link Libraries (DLLs). For this embodiment, SQL Server 2000 programming is executed by website server 24 to provide a web-based data entry/update application that maintains master data tables, permits entry of data about a patient's visit to a doctor, assigns follow-up calls to health care monitors, and facilitates entry of follow-up (outcome) information. This application permits web-based viewing of data stored in database 25 by those with corresponding privileges. In one example, the different views of the patient information provided are as follows: (a) information desired for performing follow-up calls, (b) patient outcome information, (c) patient outcome history, and (d) anonymous program information. Desired reports can be generated from the SQL database of this embodiment with Crystal Reports 8.0. Such reports can include: (a) call data to generate invoices for the monitoring service, (b) call data to generate payments to participating health care monitors, (c) results of drug outcome studies where applicable, and (d) data analysis, to name just a few examples.

This SQL embodiment of database 25 is described in terms of a number of browser-based screens. These screens are presented in a Graphic User Interface (GUI) format to provide the ability to add, change, delete, and view data. Each screen operates as a separate “window” and is based on a corresponding interface form. These forms are generated as part of the database application programming and correspond to one or more data tables comprising database 25 for this embodiment. To improve ease of use, these screens can be arranged to include a number of common elements such as a page header, menu, fields, text boxes, controls, and the like. Tools to analyze data, generate reports, and/or search/filter patient data records can also be included.

For this particular embodiment, the application commonly starts the same for all different user groups, with entry of a user identification (user id) and password at a sign-in screen followed by a legal notice of terms and conditions. A “click-on” GUI button can be provided for a user to indicate acceptance before permitting the presentation of other group or user specific screens.

After these common screens, the screen arrangement is based on the user group. Starting with administrators, a number of maintenance screens are provided. These maintenance screens can each include a menu to provide access to all other maintenance screens, which are presented in logical categories. An administrator-user can go to any screen from any screen by choosing the destination on the menu without returning to a home page to pick the new destination. While this menu can be arranged to contain every maintenance screen in the system, every user does not necessary have access to the complete array of screens, depending on security restrictions. A user id can be used to determine what, if any, restrictions might apply. The administrative menu selections can be grouped as follows:

(A) System Administration—screens to maintain users, user security, and system codes;

(B) Program Administration—screens to maintain programs, sponsors, practices, practitioners (health care providers), health care monitors, and patients;

(C) Intervention—screens used during the daily administration of patient interventions, contacts, and calls, including the assignment of calls to a health care monitor;

(D) Inquiries—screens to provide a read-only view of the data (not typically used for maintenance);

(E) Reports—screens used to define report parameters and run reports, including printing reports;

(F) Processes—screens to perform a process on a set of data (not typically used for maintenance); and

(G) Go—provides a list of maintenance screens that are related to the current screen.

From the common sign-in and legal notice screens of one SQL-implemented embodiment, an administrator is next presented a home page with the access menu. A few examples of screen selections that can be made available to an administrator through this menu are next described.

The System Administration menu group can include a “user maintenance” screen to set-up and maintain various users of the medical service website. This screen can be used to specify each user as one of four types: administrators, program sponsor, practitioner (health care provider), and health care monitor (such as a pharmacists). In another example, one or more maintenance screens are provided to define and update access privileges to some or all of the information stored in database 25. The System Administration maintenance screens can also include a “code table maintenance” screen to maintain code table entries. This screen is used to define different code types and any valid values that can be associated with each code type. Also a “contact outcome maintenance” screen can be included to view the results of a call to a patient and maintain the outcome data. In one form, this screen permits outcomes to be altered in some respects, but not to be added or deleted. This screen indicates whether or not the call is successful and includes recorded outcome data for a successfully completed call. This outcome data corresponds to patient answers to the questions asked by the health care monitor for the specified program during operation 136 of process 120. (See the Program Question Maintenance screen, which is used to define the questions to be asked for a program.). The maintenance screens can also include a dedicated screen to maintain industry standard ICD9 diagnosis codes and descriptions, and/or a screen dedicated to maintenance of industry standard NDC drug codes and descriptions.

The Program Administration menu group can include a “program maintenance” screen to maintain information descriptive of a given program, including the identification of an associated sponsor. This screen can be used to provide billing information for the program and/or other parameters that define the program, such as starting and ending dates. The Program Administration menu entries can further list maintenance screens directed to data about patients. One example is a “patient maintenance” screen to maintain general information about the patient. When a patient is involved with multiple practices, a single identifier can be used in database 25. Typically, patient data is collected the first time the patient visits a practice, and is updated any time a change is recorded during a practice visit. Another example is a “patient/program maintenance” screen that can be used to assign a patient to a particular program and maintain data about that patient specific to the program. This screen can further provide a cross-reference between patients and programs.

Program Administration screens can also be directed to the maintenance of information about health care monitors available for operations 134, 136, 138, and/or 139 (such as pharmacists). A “health care monitor maintenance” screen can be provided to maintain information that describes a given health care monitor, and can also list any programs for which the health care monitor will make calls. A “health care monitor/program maintenance” screen can also be provided that assigns a health care monitor to a program. This screen can be used as a cross-reference between health care monitors and programs. In a further example, a “health care monitor schedule maintenance” screen can be utilized to maintain health care monitor schedules. This screen can include the ability to specify the days and number of hours the health care monitor is available to make calls to patients, which can be used by any process that assigns patient calls.

The Program Administration menu group can also include a “program question maintenance” screen to define a series of questions to be asked by the health care monitor for the given program as part of operation 136, and may include parameters to specify the appearance and editing of data provided in response to such questions. The data entered with this screen can be used to generate the question fields on other screens, such as a call outcome screen. A “sponsor maintenance screen” can also be provided in this menu group to describe program sponsors. This screen can list each program associated with a given sponsor, providing the ability to drill down and view the program details, and add programs.

Program Administration maintenance screens are also directed to medical practices. A “practice maintenance screen” can be provided to maintain information descriptive of a practice, such as a name, address, phone number, fax number, etc. This screen can also provide the ability to associate one or more locations and health care providers with the practice. Patient interventions can be tracked by practice such that all or designated health care providers associated with the practice can view intervention outcome data for any patient of the practice. A “practice/location maintenance” screen can be used to link one or more locations to a practice. A “practice/practitioner maintenance” screen can be used to link a practitioner to one or more practices, and to note a primary location for the practitioner to receive outcome results under operation 139 and/or 140. A “practitioner maintenance” screen provides the ability to maintain information that describes a practitioner—name, type of health care provider, specialty (if any), DEA number, etc.

Among the Intervention menu options available to administrators for this embodiment is an “intervention maintenance” screen to record initial information about an intervention for a patient that is provided by a practice. After a patient visits the practice, the intervention information sent by the health care provider in operation 132 typically becomes the trigger for a phone call to the patient (or multiple phone calls, depending on the program) by an assigned health care monitor (operation 136). In one arrangement, the intervention maintenance screen shows the diagnosis, a list of any drugs that are prescribed, and a list of contacts for the intervention, with the ability to drill down the contact list to view the call(s) made for a contact, and the results of the call. This contact list can include both planned and completed contacts.

The Intervention menu entries also can include an “intervention contact maintenance” screen to create and maintain patient contacts for an intervention on a case-by-case basis, permitting a manual change in assignment of a health care monitor to a given patient contact. In one arrangement, this screen shows a list of the calls made for the patient contact, and provides the ability to drill down and view the details of the corresponding call. An “intervention diagnosis maintenance” screen can be used to create and maintain diagnoses for an intervention when the ability to track and report interventions by diagnosis is desired. The diagnoses can be based on industry-standard ICD9 diagnosis codes. An “intervention prescription maintenance” screen can be used to create and maintain prescriptions for an intervention when the ability to track and report interventions by drug is desired. The prescriptions can be based on industry-standard NDC drug codes.

The Intervention menu group can also include a “program contact interval maintenance” screen to define a series of patient contacts, spaced at predetermined intervals of time after the office visit. In other cases, this screen can be used to schedule a single contact a predetermined number of days after the visit for each patient contact in a given program. The entered scheduling parameters can be used to automatically generate the contacts for an intervention.

The administrator Inquiry menu options can include a “call history inquiry” screen that provides the ability to view a list of patient calls that meet specified search/filter criteria. From the list of calls, the administrative user can select a call and view details about it. The criteria to which this inquiry is responsive can include practice, date, pharmacist (or health care monitor), etc. The inquiry screens can also include a “contacts not called inquiry” to view a list of contacts that have not yet been called. The search/filter criteria used for the “call history inquiry” can also be used to retrieve records for the “contacts not called inquiry.” A “call outcome inquiry” can be provided to administrators to present a read-only view of the data recorded for a phone call to a patient by a health care monitor. In one arrangement, this screen is not accessed directly from the administrator's menu, but instead is selected from the “call history inquiry” screen by viewing a list of calls to patients, and selecting one to review in detail.

The Administrative Process menu options for this embodiment include an “assign contact” screen to automate assignment of contacts to health care monitors in operation 132 of process 120. When a contact is assigned, it will appear in the list of phone calls on the health care monitor's home page. An algorithm to assign calls can be used that takes into account a number of factors, such as the monitor's capacity, any program restrictions, number of expected patient follow-up questions, and the like. The assignment algorithm can also be responsive to administrative input parameters regarding the type or identity of the health care monitor assigned (such as a “pharmacist only” assignment), a specified contact date/time, a maximum per monitor call limit, etc. This screen or a different screen can be provided to assign a series of contacts in accordance with one or more parameters or a separate screen can provide a “contact auto-generate” process. The administrative process screens can also include a “cancel contact process” screen to execute a process to cancel contacts before they have been completed. This process can include the ability to cancel a series of contacts, facilitating, for example, the cancellation of all planned contacts for a program that has been discontinued. The cancel contact process can be responsive to one or more of the various parameters used to specify contacts, and can include the ability to edit a cancellation list. An “unassign contact process” screen may be included that automates the process of removing contact assignments from health care monitors, and deleting such assignments from the list of phone calls on the health care monitor's home page. This process can be responsive to one or more entered parameters, such as a program identifier, a given health care monitor identifier, a given practice identifier, and the like to select which contacts should be unassigned and/or from whom they should be unassigned.

The Reports menu group includes screens to determine report criteria, such as a “health care monitor payment report criteria” screen to enter criteria for printing a report that lists payments for health care monitors. This report can be used to show the amount to pay each health care monitor for contacts that were completed during a specified date range. Another can be a “program billing report criteria” screen to enter criteria for printing a list of charges that should be billed to program sponsors.

Besides the administration group, this embodiment also provides browser-based screens to health care monitors for viewing at a corresponding site, such as site 36. These screens can be used to facilitate calls to patients, showing pharmacists who they are to call, the patient's call history, and providing for the entry of data regarding the new call to the patient. Correspondingly, these screens can assist with the execution of operations 134, 136, 138, and/or 139 of process 120. A “health care monitor home page” screen can be displayed in response to log-in of a health care monitor. This page can display the calls made to patients by the respective health care monitor. A “call history list” screen to indicate all calls to a specified patient can also be included. This screen assists the health care monitor in preparing for patient calls. A “call outcome” screen can be made available to health care monitors to view the results of a previous phone call to a patient. If the call was successful, all of the patient's answers to the program questions can be displayed with such a screen. A “contact outcome entry” screen can be included that provides the ability to enter information about a phone call made to a patient. The health care monitor can record details of the call, such as whether or not it was successful. If the call was successful, the patient is asked a series of questions defined by the given program and the answers are recorded with this screen. After the information is entered and saved for a successful call via computer network 28, the data can be sent to the practice per operation 140 of process 120.

Process 120 can further be implemented by providing various browser-based screens to program sponsors. If the sponsor has more than one program, a program list can be provided upon sponsor log-in to select a program for which data is to be presented. If there is only one program for a given sponsor, data for that program is provided directly. A “sponsor home page” screen can be provided for each of the sponsor's programs. This home page can include one or more inquiries and/or reports customized to the program.

For this embodiment, several screens can be provided to practitioners to view patient outcome data as part of operations 139, 140, and/or 142. A “practitioner home page” screen can be used to access patient inquiry screens for a specified practice. This practitioner home page can provide links to inquiry screens directed to patient outcomes, and can be configured to vary among different practices. A “practice list” screen can also be included to select the practice for which they want to view patient data when the accessing practitioner is associated with more than one practice. A “patient inquiry” screen can be provided to search for a specific patient. Once the patient is located, the practitioner can choose to view a list of calls made to the patient. A “patient outcome list” screen can be provided to display all of the calls made to a patient specified with the patient inquiry screen. The practitioner can select any call in the list and view the detailed results of that call. An “outcome results” screen can be used to view the results of a phone call to a patient. If the call was successful, all of the patient's answers to the program questions can be displayed through such a screen.

It should be understood that more or fewer screens, and/or a different user interface technique could be used in other embodiments. Further, different implementation software, and/or different communication techniques may be utilized to perform process 120. Indeed, as an addition or alternative to a web-based implementation, process 120 can be implemented without the internet or world wide web, using appropriate software and hardware. For example, a windows based program can be utilized to perform process 120 over a dedicated computer network and access system. Further, in alternative embodiments, voice-over-IP, another voice communication technique using a digital computer network, a multimedia communication network, and/or email can be used in place of phone or fax communications. In one alternative embodiment, a voice and/or video recording of a patient contact by a health care monitor is generated and sent via computer network 28 to database 25 for storage as part of operations 136 and/or 138.

Many other embodiments of the present invention are envisioned. For example, in one embodiment of the present invention an internet-based system is provided that will enable doctors and pharmacists to obtain and share information about patients' adherence to prescribed medications by linking physicians and pharmacists in the evaluation of patient compliance and the success of therapy. The resulting data can be used to adjust therapy on a timely basis and provide outcome information for subsequent office visits. This procedure can improve patient satisfaction and lower health care costs through better and more efficient utilization of medication.

A further embodiment of the present invention includes: contacting a patient after a visit to a health care provider to obtain post-visit information; entering the post-visit information into patient information database through a computer network; and providing at least a portion of the post-visit information to the health care provider from the database through the computer network. In one form, the patient contact is made by a pharmacist in response to an assignment provided over the computer network from a remotely located administrative site. This assignment can be generated by a server coupled to the computer network in response to a request by the health care provider. The patient contact can include a number of program-specific questions posed to the patient. Alternatively or additionally, the patient information database is maintained by a monitoring service, and is remotely located relative to the patient, the health care provider, and the party contacting the patient.

Another embodiment comprises: providing a server for storing a patient information database; assigning a patient contact to a health care monitor through a computer network coupled to the server; receiving a patient response obtained by the health care monitor through the computer network; storing the patient response received from the health care monitor in the patient information database; and sending data about the patient response to a health care provider from the patient information database. In various forms of this embodiment, the health care monitor is a pharmacist, the health care monitor is remotely located relative to the server, the health care monitor assignment is generated by the server, and/or the health care monitor contacts the patient by telephone. Alternatively or additionally, this embodiment can include: providing an administrative site coupled to the server by the computer network, generating the health care monitor assignment in response to a request from the health care provider, entering patient data into the patient information database in response to a follow-up request from the health care provider, and/or providing patient data to a program sponsor through the computer network.

Still another embodiment includes: providing a server for storing a patient information database; generating an assignment of a patient contact to a health care monitor with the server; sending the assignment to the health care monitor through a computer network coupled to the server; receiving patient data through the computer network from the health care monitor, the patient data being provided in response to the patient contact by the health care monitor; storing the patient data received from the health care monitor in the patient information database; and sending the patient data to a health care provider for the patient from the patient information database. In some forms of this embodiment, the health care monitor is a pharmacist, the health care monitor is remotely located relative to the server, the health care monitor assignment is generated by the server, and/or the health care monitor contacts the patient by telephone. Alternatively or additionally, this embodiment can include: providing an administrative site coupled to the server by the computer network, generating the health care monitor assignment in response to a request from the health care provider, entering patient data into the patient information database in response to a follow-up from the health care provider, and/or providing patient data to a program sponsor through the computer network.

Yet another embodiment comprises: receiving a patient follow-up request from a practitioner; entering data in a patient information database maintained by a server in response to the patient follow-up request; assigning a patient contact to a health care monitor from the server over a computer network, the patient contact being assigned in response to the patient follow-up request; receiving patient data over the computer network from the patient contact performed by the health care monitor; storing the patient data in the patient information database; and sending the patient data to the practitioner from the patient information database. In some forms of this embodiment, the health care monitor is a pharmacist, the health care monitor is remotely located relative to the server, the health care monitor assignment is generated by the server, and/or the health care monitor contacts the patient by telephone. Alternatively or additionally, this embodiment can include: providing an administrative site coupled to the server by the computer network and/or providing patient data to a program sponsor through the computer network.

In another embodiment, a computer-accessible device includes programming instructions executable by a computer to perform a method of patient monitoring, the method comprising: generating an assignment of a patient contact to a health care monitor from a patient information database maintained by the server; sending the assignment to the health care monitor through a computer network coupled to the server; receiving patient data through the computer network from the health care monitor, the patient data being provided in response to the patient contact by the health care monitor; storing the patient data received from the health care monitor in the patient information database; and sending the patient data to a health care provider for the patient from the patient information database. In some forms of this embodiment, the health care monitor is a pharmacist, the health care monitor is remotely located relative to the server, the health care monitor assignment is generated by the server, and/or the health care monitor contacts the patient by telephone. Alternatively or additionally, the method can include: providing an administrative site coupled to the server by the computer network, generating the health care monitor assignment in response to a request from the health care provider, entering patient data into the patient information database in response to a follow-up from the health care provider, and/or providing patient data to a program sponsor through the computer network.

For still a further embodiment of the present invention, a system comprises: a server operable to maintain a patient information database; an administrative computer; one or more practitioner computers; one or more health care monitor computers; a computer network coupled to the server, the administrative computer, the one or more practitioner computers, and the one or more health care monitor computers. The server is operable to assign one or more patient contacts to a health care monitor through one of the one or more health care monitor computers over the computer network, receive a patient response obtained by the health care monitor through the computer network, store the patient response in the patient information database received from the health care monitor, and send data about the patient response to at least one of the one or more practitioner computers from the patient information database. In one form of this embodiment, a program sponsor computer is also coupled to the computer network.

Any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein are desired to be protected.

What is claimed is:

1. A method, comprising:
providing a server coupled to a computer network;
receiving a patient follow-up request from a practitioner;
entering data in a patient information database maintained by the server in response to the patient follow-up request;
assigning a patient contact to a health care monitor from the server over a computer network;
receiving patient data over the computer network from the health care monitor about the patient contact;
storing the patient data in the patient information database; and
sending the patient data to the practitioner from the patient information database.

2. The method of claim 1, wherein the health care monitor is a pharmacist.

3. The method of claim 1, wherein the health care monitor is remotely located relative to the server, and said assigning includes designating several different patient contacts for the health care monitor from the server over the computer network.

4. The method of claim 1, wherein the health care monitor contacts the patient by telephone to perform the patent contact.

5. The method of claim 1, which includes:
providing an administrative site coupled to the server by the computer network;
providing at least a portion of the patient data to a program sponsor through the computer network; and
evaluating the patient data to determine patient compliance.

6. A method, comprising:
providing a server for storing a patient information database;
generating an assignment of a patient contact to a health care monitor with the server;
sending the assignment to the health care monitor through a computer network coupled to the server;
in response to the patient contact by the health care monitor, receiving patient data through the computer network from the health care monitor;
storing the patient data received from the health care monitor in the patient information database; and
sending the patient data to a health care provider for the patient from the patient information database.

7. The method of claim 6, wherein said generating is performed in response to a request from a health care provider.

8. The method of claim 7, wherein the patient receives a prescription from the health care provider and the request from the health care provider is generated to determine compliance of the patient with the prescription.

9. The method of claim 6, wherein the health care monitor is a pharmacist remotely located relative to the server.

10. The method of claim 9, which includes the pharmacist providing counseling to the patient regarding a drug treatment.

11. The method of claim 6, which includes entering a patient diagnosis and one or more drug codes into the patient information database, and designating the health care monitor to be a pharmacist.

12. The method of claim 6, which includes the health care monitor performing the patient contact by telephone.

13. The method of claim 6, which includes the health care monitor asking assigned questions of the patient during the patient contact.

14. The method of claim 6, which includes providing the patient data to a drug outcome study.

15. The method of claim 6, which includes the health care provider adjusting medical treatment of the patient in response to the patient data after said sending.

16. A computer-accessible device, comprising: logic executable by a computer server to perform patient monitoring by generating an assignment of a patient contact to a health care monitor from a patient information database maintained by the server in response to a request from a health care provider, sending the assignment to the health care monitor through a computer network coupled to the server, receiving patient data through the computer network from the health care monitor in response to the patient contact by the health care monitor, storing the patient data received from the health care monitor in the patient information database, and sending the patient data to the health care provider for the patient from the patient information database.

17. The device of claim 16, wherein the logic is in the form of programming instructions.

18. The device of claim 16, wherein the logic is in the form of one or more signals transmittable over the computer network.

19. A system, comprising:
a server operable to maintain a patient information database;
an administrative computer;
one or more practitioner computers;
one or more health care monitor computers;
a computer network coupled to the server, the administrative computer, the one or more practitioner computers, and the one or more health care monitor computers; and
wherein the server is operable to assign one or more patient contacts to a health care monitor through one of the one or more health care monitor computers over the computer network, receive a patient response obtained by the health care monitor through the computer network, store the patient response in the patient information database received from the health care monitor, and send data about the patient response to at least one of the one or more practitioner computers from the patient information database.

20. The system of claim 19, further comprising a program sponsor computer coupled to the computer network.

21. The system of claim 19, wherein the server is operable to receive information about a request from a practitioner to follow-up with the patient and generate an assignment of at least one of the patient contacts in response to the request from the practitioner.

22. The system of claim 19, wherein the computer network includes the internet, the server is of a website type, and the administrative computer, the one or more practitioner computers, and the one or more health care monitor computers operate as clients of the server.
